(19) United States
(12) Patent Application Publication
(10) Pub. No.: US 2012/0191064 A1
(43) Pub. Date: Jul. 26, 2012

(54) METHODS AND APPARATUS FOR SUB-RETINAL CATHETERIZATION

(75) Inventors: Stanley R. Conston, San Carlos, CA (US); Ronald Yamamoto, San Francisco, CA (US)

(73) Assignee: ISCIENCE INTERVENTIONAL CORPORATION, Menlo Park, CA (US)

(21) Appl. No.: 13/320,218

(22) PCT Filed: May 14, 2010
(86) PCT No.: PCT/US10/34873

§ 371 Date: Dec. 5, 2011

Related U.S. Application Data
(60) Provisional application No. 61/178,882, filed on May 15, 2009.

(51) Int. Cl. A61M 5/00; A61L 29/14; A61M 25/00

(52) U.S. Cl. 604/506; 604/523; 604/265; 604/272

(57) ABSTRACT

Devices and methods are provided for access to the sub-retinal space that lies between the retina and the choroid in order to introduce therapies to the retina and more specifically to the sensory retina and RPE, particularly in the region of the macula. The devices comprise a catheter that incorporates advantageous size, flexibility and tip features to properly, accurately and atraumatically access the sub-retinal space. Ancillary devices to assist in placing catheters into the sub-retinal space are also provided. The catheter devices incorporate a lumen for delivery of therapeutic substances or devices into the eye.

This application claims the priority under 35 USC §119(e) and §§363-365 of U.S. Provisional Application No. 61/178,882, filed May 15, 2009, the disclosure of which is incorporated herein by reference in its entirety for all purposes.

### BACKGROUND OF INVENTION

There are many diseases and conditions that affect the retina which can lead to a progressive decrease in visual acuity and eventual blindness. Deleterious consequences from disease processes or physiological defects can affect specific tissues of the retina such as the photoreceptors, ganglion cells and the retinal pigment epithelium (RPE). Diseases such as age-related macular degeneration, diabetic retinopathy, retinitis pigmentosa, Stargardt's disease and conditions such as macular holes, retinal detachments, epiretinal membranes, retinal or choroidal venous occlusions can all lead to vision loss that ranges from mild to total. Many of these ailments are treated through systemic or intravitreal injections of pharmaceutical agents, or via surgery through the vitreous cavity. New treatments using tissue transplantation or translocation and the delivery of biological agents or cells to the retina provide alternatives for many severe retinal disease conditions. Procedures such as macular translocation, RPE cellular and tissue transplants or even the placement of retinal implants are examples of new techniques and technologies that require means to access the retina and subretinal space to apply therapeutic agents or devices at specific locations.

Interventional procedures targeting tissues beneath the sensory retina are difficult to perform due to limited accessibility and the delicate structure of the retina which can be easily damaged during surgical manipulation. This is especially critical in the macular foveal region of the retina that functions to provide central and color vision. It is desired to provide devices and methods for accessing and delivering therapies in a safe manner to the macular region without forming a penetrating hole in the macula that may lead to sight threatening complications. Accessing the sub-retinal space with a catheter at a location distant from the macula and advancing the catheter in the sub-retinal space to the macula would allow for the safe and direct intervention to the sensory retina and the RPE. The present invention provides devices and methods which can provide such access to perform retinal treatment.

The devices according to the present invention are adaptable for the specific treatment being delivered. Examples of treatments include the delivery of surgical tools, pharmaceutical or biological agents, tissues or cellular grafts, transplants or implants

### SUMMARY OF THE DISCLOSURE

The present invention provides devices for access to the subretinal space of an eye comprising:
a catheter having a proximal end and distal end, said distal end comprising an atraumatic tip having a smooth surface;
the catheter having flexural rigidity in bending and a response to critical buckling load sufficient to allow flexing of the catheter in the eye without causing substantial tissue trauma or distension of local tissue.

The useful flexural rigidity in bending of the catheter is less than 2.04×10⁻⁹ kN-m². The useful response to critical buckling load of the catheter is less than 21.08 grams-force. Typically the catheter will also have a round profile of a maximum diameter of at least 200 microns.

The device may comprise an illuminated beacon tip. The surface of the catheter may be lubricious and the catheter may comprise external depth markings.

In one embodiment the catheter may comprise a region adjacent to the atraumatic tip having lower flexural rigidity than that of the catheter to allow the tip to flex upon encountering an obstruction during insertion of the catheter into the eye.

The invention also provides an auxiliary tool which may be a tubular shaft introducer characterized by a primary shaft axis and comprising a distal end disposed at an angle to the primary shaft axis, the shaft comprising a lumen of sufficient diameter to accommodate a catheter having a smooth surface and a round profile of a maximum diameter of at least 200 microns. The angle is usefully in the range of 20° to 90°. The distal end forming the angle with the primary shaft axis is usefully of a length of 2 to 10 mm. The primary shaft axis typically has a length of 25 to 40 mm.

A cannula device is also provided by the invention having a proximal end and distal end, the device comprising a bulbous distal tip and a lumen, the bulbous distal tip being of sufficient size to dissect the choroid of an eye to access the subretinal space for injecting through said lumen a viscoelastic substance to create a fistula in the subretinal space of the eye. The bulbous tip typically has a diameter of at least 200 microns. The cannula device may also have a protruding element at the distal tip. The protruding element will typically protrude from 10 to 100 microns from the distal tip. A useful protruding element is a fiberoptic.

Methods are provided for catheterizing the sub-retinal space adjacent to the macula of an eye by introducing a catheter into the sub-retinal space in an area of peripheral retina by advancing a tip of the catheter in the sub-retinal space toward the macula. In one embodiment the catheter has a proximal end and distal end and the distal end comprises the tip advanced in the sub-retinal space, the distal end comprising an atraumatic tip having a smooth surface;
the catheter having flexural rigidity in bending and a response to critical buckling load sufficient to allow flexing of the catheter in the eye without causing substantial tissue trauma or distension of local tissue.

In another embodiment of the method a cannula device is used having a proximal end, a bulbous distal tip and a lumen, the bulbous distal tip being of sufficient size to dissect the choroid of an eye to access the subretinal space for injecting through the lumen a viscoelastic substance to create an opening through the choroid into the sub-retinal space of the eye for the purposes of placing a catheter into the subretinal space of the eye.

Methods are also provided for catheterizing the sub-retinal space adjacent to the macula of an eye by introducing a tubular shaft into the sub-retinal space in an area of peripheral retina, the shaft characterized by a primary shaft axis and comprising a distal end disposed at an angle to said primary shaft axis, the shaft comprising a lumen of sufficient diameter to accommodate a catheter having a smooth surface.

Methods are also provided whereby a catheter is placed in the sub-retinal space from an ab-interno approach, by forming an opening in the peripheral retina ab-interno, to access the sub-retinal space, placing the tip of the catheter through the retinotomy and advancing it posteriorly, administering therapeutic substances, withdrawing the catheter and sealing the opening in the peripheral retina.

### DESCRIPTION OF THE DRAWINGS

FIG. 1: Schematic of catheter device.

FIG. 2: Detailed schematic of distal shaft of the catheter device.

FIG. 3: Schematic of tubular introducer device for ab-interno access.

FIG. 4: Schematic of tubular introducer access to the subretinal space ab-interno.

FIG. 5: Schematic of the catheter device deployed in the sub-retinal space through the introducer.

FIG. 6: Schematic of viscodissection cannula.

FIG. 7: Schematic of viscodissection cannula with a protruding element disposed at the distal tip.

FIG. 8: Schematic of viscodissection cannula creating access to the sub-retinal space.

FIG. 9: Schematic of catheter device ab-externo access through the sclera and choroid and into the sub-retinal space.

### DESCRIPTION OF THE PREFERRED EMBODIMENTS OF THE INVENTION

The present invention provides devices and methods for access to the sub-retinal space that lies between the retina and the choroid in order to introduce therapies to the retina and more specifically to the sensory retina and RPE, particularly in the region of the macula. The devices comprise a catheter that incorporates the appropriate size, flexibility and tip design to safely access the sub-retinal space. Ancillary devices to assist in placing catheters into the sub-retinal space are also provided. The catheter devices incorporate a lumen for delivery of therapeutic substances or devices. The catheters may incorporate a light source or markings to aid visualization of the catheter to guide the surgeon during placement and advancement.

The present invention provides devices and methods for access to the sub-retinal space in order to deliver devices, materials, energy or substances to the adjacent tissues. In addition, the invention provides devices to access the sub-retinal space at the peripheral retina, place a catheter into the sub-retinal space and advance the catheter tip in the sub-retinal space to the macular region of the retina.

In catheterizing the sub-retinal space, minimizing trauma is important, as tearing of the overlying retina or damage to the underlying RPE can result in significant loss of vision, especially in the area of the macula where treatment is most beneficial. Atraumatic characteristics of the devices of the present invention are provided by one or more elements, including selected mechanical properties, tip design, the use of friction minimizing tissue contact surfaces, and incorporation of guidance components on the catheter device. The sub-retinal space is a space between spherical tissue planes and is not a tubular tract such as a blood vessel or Schlemm's canal of the eye where the walls of the tract provide mechanical support during catheter advancement. Due to the lack of lateral constraint during catheter advancement, the mechanical properties are selected to provide a proper response to both flexural (bending) and axial loading anticipated to be encountered by using the devices and methods according to the present invention. These characteristics are useful in the treatment of diseased eyes in the sub-retinal space, where lesions, hemorrhage or scarring from previous treatment(s) may act to deflect a catheter during advancement, potentially causing tissue damage. The ancillary devices provided by the invention allow introduction of the catheter into the sub-retinal space with minimal trauma and in a fashion to guide the catheter along the plane of the sub-retinal space.

The present invention provides a catheter device with an atraumatic tip among other advantageous features. The tip comprises a rounded profile and smooth surface to prevent trauma to, and penetration of, the retina. A useful embodiment of an atraumatic tip has a smoothly radiused, bulbous or olivary shape where the diameter of the tip is larger than the diameter of the shaft of the catheter. A rounded tip having a diameter of at least 200 microns (0.008 inch) is particularly useful to limit penetration through the sensory retina.

In addition to the atraumatic tip, a useful feature is to provide the catheter with mechanical properties that allow the retina contacting distal portion of the catheter to follow the curvature of the eye, minimizing distension of the adjacent tissues and the potential for localized tissue trauma, especially the thin and delicate sensory retina. The retina contacting distal portion length is in the range of 25 mm (1 inch)-40 mm (1.6 inch). Such mechanical properties include both the appropriate bending resistance or flexural rigidity, of the catheter along the length of the catheter shaft that is being advanced and the force required to cause flexing, the critical buckling load, of the distal end of the catheter upon axial loading. A flexural rigidity in bending of less than 2.04×10⁻⁹ kN*m² and a critical buckling load of less than 21.08 grams-force are useful for safe catheterization of the sub-retinal space.

An embodiment to further provide improved atraumatic catheterization of the sub-retinal space incorporates a hinge-like region at or near the transition of the catheter shaft to the atraumatic tip to reduce the critical buckling load at the distal tip. A flexural rigidity of the hinge region that is less than that of either the adjacent atraumatic tip or the adjacent catheter shaft allows the atraumatic tip to flex when encountering an obstruction such as an area of fibrosis. The resulting tip deflection is an improvement to act to direct the catheter around the obstructed area to avoid potential mechanical trauma.

The catheter may also be sized appropriately to minimize the fluid volume of the device to aid in the delivery of small quantities of substances. A catheter providing total luminal volume in the range of 100-250 microliters is appropriate for the delivery of such quantities of fluid substances. The catheter may also be provided with luminal passages and transitions between tubular segments that are smooth in order to minimize the shear forces on the agent being delivered, which is advantageous in the delivery of biologic agents. Catheters having an inner diameter in the range of 100 microns (0.004 inch) to 250 microns (0.010 inch) with a wall thickness of 25 microns (0.001 inch) to 50 microns (0.002 inch) are particularly useful. The catheter may comprise a variety of flexible polymers including polysiloxanes, polyurethanes, polyether block amides (PEBAX), polyalkanes, fluoropolymers, polyamides, polyethylene terephthalates, and combinations of such polymers.

The catheter may be provided with a coating, markers, or a light source at or near the tip aid in catheterization and to identify the tip location during surgical placement to the desired location. The coating or markings may comprise opaque inks, or other optically visible elements; radio-opaque, radio-frequency, ultrasound interacting, infrared, or other non visible identification elements, attached or integrated into the catheter. The coating may comprise a hydrophilic or lubricious material to aid in catheterization and reduce friction with contacting tissues. The light source may comprise a fiber optic element that conducts light to the atraumatic tip to provide a visible light beacon to readily identify location of the tip. The distal end of the fiber optic is preferred to be located within or just proximal to the atraumatic tip to allow the tip to distribute the light in a lateral direction to aid off-axis visualization.

The catheter may be introduced into the sub-retinal space either ab-interno methods from within the vitreous cavity or ab-externo methods by way of a scleral dissection to the sub-retinal space.

In the ab-interno approach, the catheter is passed into a small retinotomy incision, or opening, made in the peripheral retina. A small tubular introducer with a curved or angled distal end may be used to introduce the catheter into the sub-retinal space and then to direct it parallel to the retinal surface to the macula. The introducer is typically sized to allow the catheter device to fit slideably within the introducer lumen. It is useful to provide an introducer having an outer diameter sized in the range of 0.5 mm (0.020 inch) to 0.9 mm (0.036 inch) to fit through standard sclerostomy ports, which are typically sized at 20, 23 or 25 gauge. The distal tip of the introducer should be disposed at an angle from the main introducer shaft axis, with a smooth transition between the shaft and the angled tip to allow unimpeded passage of the catheter through the introducer. A tip angled in the range from 20° to 90° from the main axis is useful to allow for entry into the sub-retinal space. The length of the distal tip is usefully in the range of 2 mm (0.08 inch) to 10 mm (0.40 inch). The introducer main shaft will usefully have a length from 25 mm (1.0 inch) to 40 mm (1.6 inch). The distal tip may be beveled for ease of access to the sub-retinal space. The introducer may comprise rigid materials including metals, polyetheretherketone (PEEK), polyethylene, polypropylene, polyimide, polyamide, polysulfone, polyether block amide (PEBAX), fluoropolymers or combination of such materials. The retinotomy incision may be sealed after completion of the treatment and removal of the catheter, either with a laser, diathermy probe or a cryoprobe. The catheter may also be used to introduce a tissue sealant as it exits the site.

An ab-interno method may also be used whereby a catheter is placed in the sub-retinal space from an ab-interno approach, by forming an opening in the peripheral retina ab-interno, to access the sub-retinal space, placing the tip of the catheter through the retinotomy and advancing it posteriorly, administering therapeutic substances, withdrawing the catheter and sealing the opening in the peripheral retina.

In the ab-externo approach, the sclera at or slightly posterior to the pars plana region over the peripheral retina is dissected to expose the choroid. The sclera is dissected to access the suprachoroidal space between the sclera and choroid to expose the choroid. As the choroid is a highly vascularized tissue, it is desired to be able to create atraumatically a fistula or opening through the choroid for catheter access to the underlying sub-retinal space. Viscodissection or fluid dissection is the separation of tissues or tissue planes using a viscoelastic or fluid. A cannula device comprising a fine gauge cannula, with a bulbous distal tip, a proximal Luer fitting and a small diameter distal lumen may be used to inject a fluid or high viscosity viscoelastic substance directly onto the choroidal surface in order to gently dissect the tissue and create an opening to the sub-retinal space. A bulbous tip of at least 200 micron (0.008 inch) diameter is useful to minimize perforation of the retina. The main shaft of the viscodissection cannula may be straight or the distal section may be angled to allow for better visualization of the distal tip during the procedure. The angle may be in the range of 30° to 60° and typically is 45°.

A particularly useful device for an ab-externo approach is a viscodissection cannula with a protruding element at the distal tip that has a small cross-sectional dimension and extends past the distal end of the lumen. The element acts to pierce the choroid and aids the effect of the viscoelastic substance to dissect in the direction of the protruding element. The cannula may optionally incorporate a tube forming the distal tip, the tube comprising a thin walled metal or plastic such as polyimide and have an inner diameter in the range of 25 microns (0.001 inch) to 150 microns (0.006 inch) and a wall thickness from 10 microns (0.0004 inch) to 100 microns (0.004 inch). The tube may be disposed within the outer tube of the cannula, which may be metallic tubing sized in the range of 25-32 gauge which furthermore incorporates a Luer connector at the proximal end for the introduction of fluids and viscoelastics. The thin walled tube may extend beyond the distal tip of the cannula shaft for a distance in the range of 25 microns (0.001 inch) to 100 microns (0.004 inch). The protruding element may comprise a metallic wire, such as stainless steel, nitinol or tungsten, in a diameter between 10 microns (0.0004 inch) and 100 microns (0.004 inch), disposed within or adjacent to the lumen of the thin walled tube. The protruding element typically may extend beyond the distal end of the lumen for a distance in the range of 25 microns (0.001 inch) to 75 microns (0.003 inch). The protruding element preferably incorporates a beveled or sharp distal tip to aid in piercing the tissues. In another embodiment, the distal end of the thin walled tube may be formed to incorporate an integral protruding element, for example by cutting the tip so as to leave a sharp or triangular shaped point extending from the edge. The protruding element extends beyond the distal edge of the tube a distance between 25 microns (0.001 inch) and 75 microns (0.003 inch). In another embodiment, the protruding element may comprise a fiberoptic fiber to allow visualization ab-interno of the position of the distal end of the dissection tool during penetration of the choroid to avoid damage to the retina.

The catheter is advantageously inserted through the choroidal opening and into the sub-retinal space and then advanced posteriorly toward the macula. A tubular introducer as previously described to guide the catheter during insertion into the sub-retinal space during the ab-interno approach may be first placed into the choroidal opening to aid proper and accurate catheter placement and minimize the potential for inadvertent penetration of the retina during catheter advancement. The tip of the catheter may be observed with a surgical microscope or indirect ophthalmoscope through the pupillary aperture during catheter advancement. With the ab-externo catheterization approach, no hole is made in the retina, reducing trauma, the potential for endophthalmitis and possible leakage of the injected substances into the intraocular space.

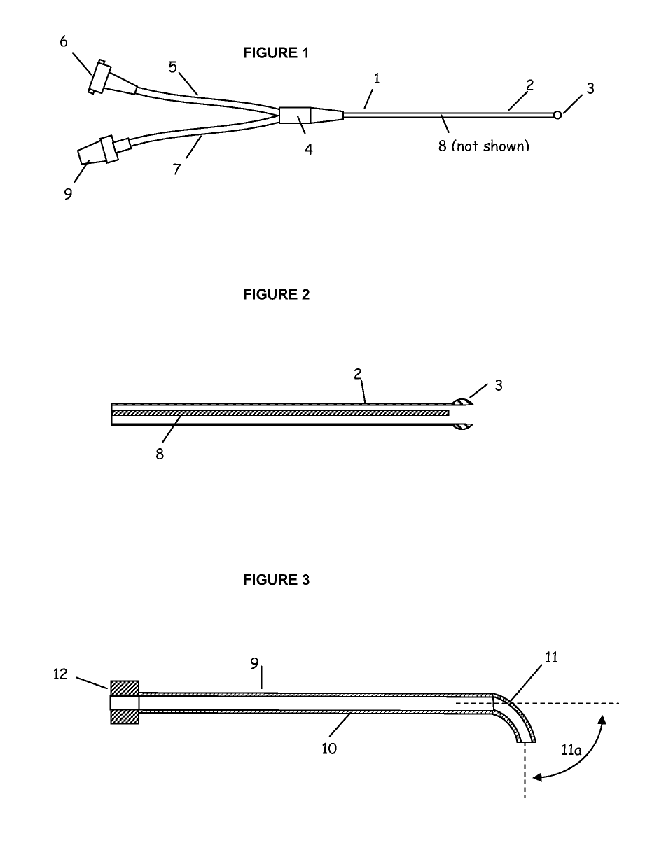

As shown in FIG. 1, the catheter device 1 comprises a tubular distal member 2 suitably sized to access and traverse the subretinal space in an atraumatic manner. The tip 3 of the distal member is shaped into a smoothly radiused tip of larger diameter than the tubular shaft. The distal member is connected through a hub element 4 and is in communication with at least one proximal tubular member. In a preferred embodiment comprising two proximal members, one proximal member comprises a tube 5 and an terminating fitting 6 such as a Luer connector which is in communication with the lumen of the distal member 2 and to which a device such as a syringe may be attached; and a second proximal member comprising a flexible fiberoptic 7 connected to a fiberoptic 8 residing in the lumen of the distal member 2 and terminating proximally in a fiberoptic connector 9 for attachment to a light source.

FIG. 2 shows a detailed view of the distal tubular member 2, terminating in a smoothly radiused tip of larger diameter 3 and a flexible fiberoptic 8 residing in the lumen.

FIG. 3 shows a detailed view of a tubular introducer device 9 for ab-interno access to the subretinal space. The introducer is comprised of a thin walled tubular shaft 10, a curved distal tip 11 disposed at an angle 11a to the main shaft and a proximal hub 12. The inner diameter of the introducer is sized to allow the catheter device to slideably fit within. The distal tip 11 may be usefully curved in the range of 20° to 90°.

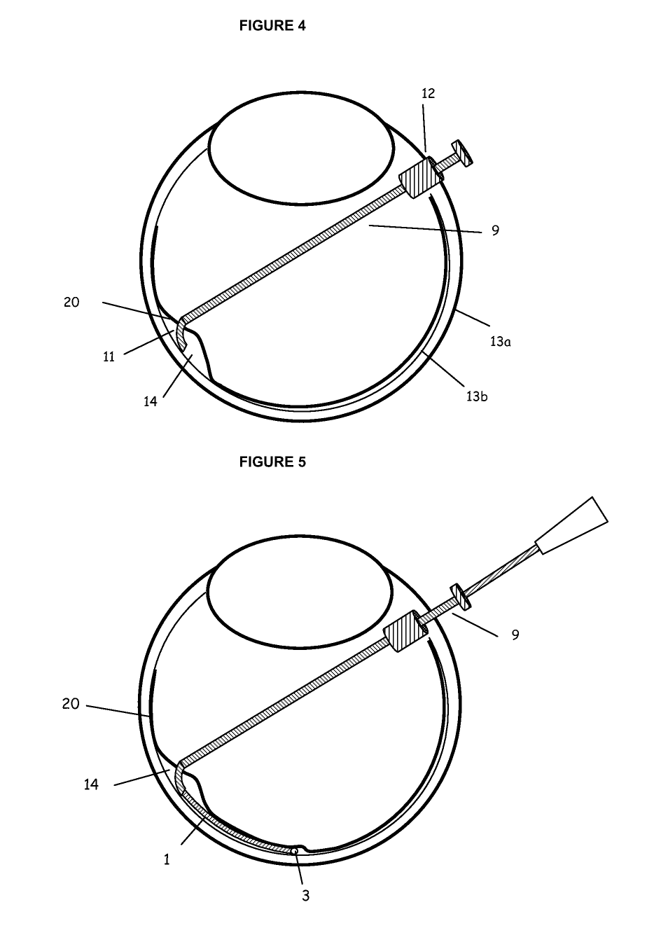

The use of a device according to an ab-interno approach is detailed in FIGS. 4 and 5. FIG. 4 shows the tubular introducer 9 with a curved distal tip 11. The introducer is placed through a sclerostomy port 12 which has been inserted through the sclera 13a and choroid 13b at the pars plana. The introducer is advanced across the globe. The curved distal tip 11 of the introducer is inserted through the retina 20 providing access to the sub-retinal space 14. FIG. 5 shows a catheter device 1, placed through the introducer 9 and advanced in the sub-retinal space 14 until the distal tip 3 resides under the macula.

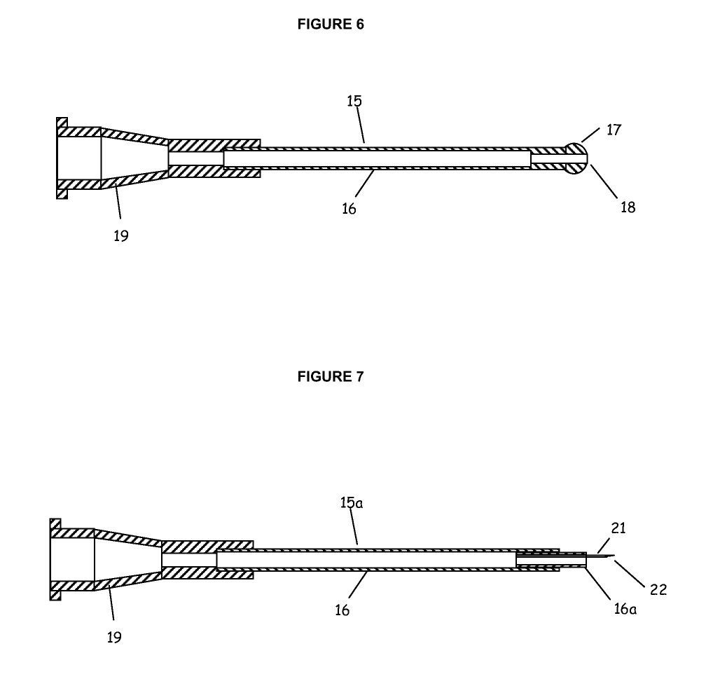

FIG. 6 shows a detailed view of the viscodissection cannula 15. The cannula comprises a rigid shaft 16 composed of metal or plastic with a bulbous rounded distal tip 17 and a small diameter distal lumen 18. The proximal end of the cannula comprises a Luer fitting 19 for attachment to a fluid dispensing device such as a syringe.

FIG. 7 shows a detailed view of the viscodissection cannula 15a with a protruding element 21 The cannula comprises a rigid shaft 16 composed of metal or plastic with a thin walled tube 16a disposed within the distal tip of the rigid shaft. A protruding element 21 with a beveled tip 22 is disposed within the thin walled tube and extends beyond the tip of the tube. The proximal end of the cannula comprises a Luer fitting 19 for attachment to a fluid dispensing means such as a syringe.

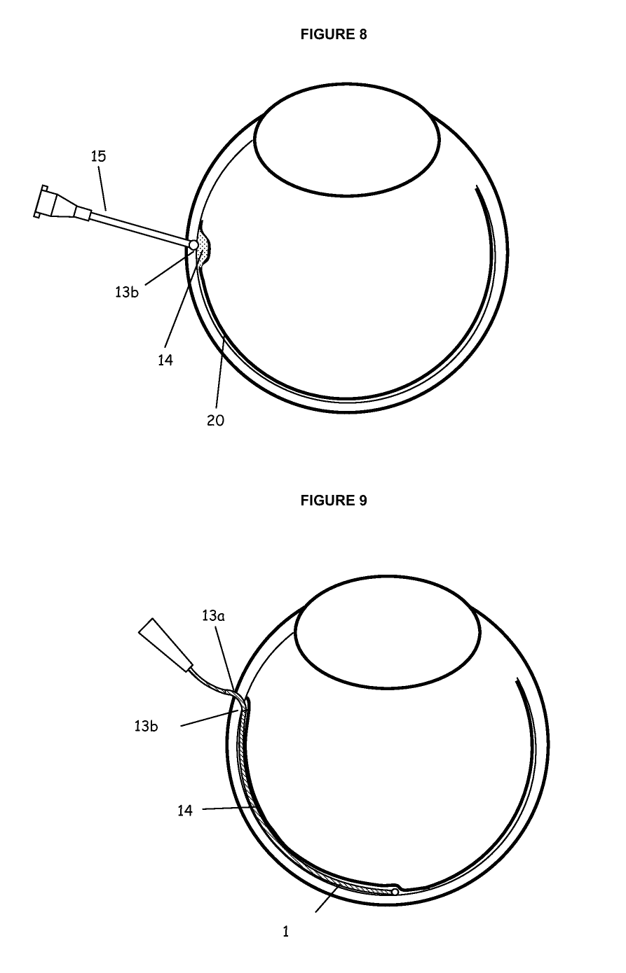

FIG. 8 shows the viscodissection cannula 15 creating a fistula through the choroid 13b while a high viscosity viscoelastic is injected. As the viscodissection cannula 15 advances through the choroidal tissue, the viscoelastic being expressed from the tip creates a small pocket or bleb in the sub-retinal space 14 under the retina 20 and allowing for access to the sub-retinal space.

FIG. 9 shows use of a device according to an ab-externo approach wherein the catheter device 1 has been inserted through an incision in the sclera 13a, through a fistula in the choroid 13b, and advanced along the sub-retinal space 14.

The following examples are provided for the purpose of illustration. These examples not intended to limit the scope of the invention in the appended claims in any way.

### EXAMPLES

### Example 1

Two enucleated rabbit eyes and a human cadaver eye were prepared for testing. The pars plana region of the sclera was dissected with an approximately 4 mm incision to expose the choroid. A series of rounded steel probes were used to apply pressure on the choroid and retina to determine if a particular size range of an atraumatic catheter tip would help to prevent inadvertent penetration into the posterior chamber.

Rounded steel probes with the tip diameters as shown in Table 1 were tested during dissection of the choroid to the sub-retinal space. Table 1 also shows the resultant effects observed.

TABLE 1
Probe tip diameters and dissection results

| Probe tip diameter | Effect on choroid and retina |
|---|---|
| 115 microns | Easily penetrates into posterior chamber |
| 165 microns | Easily penetrates into posterior chamber |
| 220 microns | Blunt dissects the choroid, must be careful |
| | not to penetrate into posterior chamber |
| 275 microns | Blunt dissects the choroid |
| 330 microns | Blunt dissects the choroid |
| 360 microns | Blunt dissects the choroid |
| 415 microns | Difficult to penetrate choroid and retina |
| 460 microns | Difficult to penetrate choroid and retina |

Results from enucleated rabbit eyes and a human cadaver eyes demonstrates that a rounded tip less than 220 microns in diameter easily penetrates into the posterior chamber and does not provide a guard from penetration through the retina.

### Example 2

Enucleated human cadaver eyes were used to determine the mechanical properties for atraumatic advancement in the sub-retinal space. The eyes were prepared in an “open sky” approach by dissecting off the anterior segment of the globe at the level of the ciliary body, and removing the lens. In a living eye, the retinal tissues are attached to the RPE by interdigitation of the cells and the fluid pumping mechanism of the RPE. Post-mortem, the retina no longer has strong attachment to the RPE, so a method was used to maintain the positioning of the retina during the experiments. A heavy fluid, perfluoromethylcyclopentane (Flutec PC1C, F2 Chemicals LTD), with a density of 1.707 Kg/L was injected into the vitreous cavity to displace the vitreous fluid and to hold the retina in place similar to the use of heavy fluids in retinal detachment repair. A notch was cut into the globe down to the level of the anterior insertion of the retina, in order to gain direct access to the retina from an ab-interno approach.

Mechanical models of sub-retinal catheters of the present invention were prepared from metal wires composed of 304 stainless steel and Nitinol (nickel titanium alloy) (Ft. Wayne Metals, Inc) of various diameters. The wires were in their cold worked (as drawn) condition and the nominal modulus of elasticity (E) for the stainless steel wires was 196 GPa and the Nitinol wires was 41 GPa. The wire ends were rounded using a YAG laser to create atraumatic tips nominally 2-3 times the diameter of the wire models. In the experiment, the wires were sequentially placed under the retina at the anterior insertion, and then advanced toward the posterior pole and optic nerve. Each test was visually scored, with a test sample being able to advance to the posterior pole with minimal displacement or “tenting” of the overlying retina (<2 mm) along the tract scored as passing, and if the test sample was not able to be advanced or had observed deformation of the overlying retina, it was scored as a failure.

Each wire sample was evaluated using a mechanical tester (Instron) with a 5 Newton load cell to determine it's flexural rigidity by 3-point bending and critical buckling load by axial compression. Flexural rigidity in bending and critical buckling loads were calculated from the output of the Instron. The bending modulus, E_{B}, was determined by using a modified ASTM D790-07 Flexural Test method. Due to the very small diameter of the wire samples, the test method was modified by using smaller supports and a loading nose of 0.095 inch (2.4 mm) diameter and a smaller support span of 0.200 inch (5.08 mm). The Instron result of E_{B} was then multiplied by the 2^{nd} moment of inertia, I, to yield the flexural rigidity, E*I. The moment, I, was calculated using I=π*r²/4, where r equals the radius of the sample. The mechanical models were of precise geometry and construction to yield results of high accuracy. Five samples of each mechanical model were tested by the 3-point bending method.

Critical buckling loads were determined using the ASTM E9-09 Compression Test method. The critical buckling loads were measured directly from the output of the mechanical tester for each sample. Ten samples of each mechanical model were tested by the compression method.

Table 2 shows the range of wire types and sizes, the measured flexural rigidity, the measured critical buckling load and the test results of the in-vitro sub-retinal passage. Note that the flexural rigidity of the 0.001″ diameter Nitinol was not determined due to its low deflection force being below the sensitivity of the load cell.

TABLE 2
Mechanical Models—Measured Properties and In-Vitro Results

| | | | Critical | In-Vitro |
|---|---|---|---|---|
| | | Flexural | Buckling | Test |
| | Diameter inch | Rigidity | Load | Results |
| Wire Material | (mm) | (kN*m²) | (gf) | (Pass/Fail) |
| Nitinol | 0.001 (0.025) | N/A | 0.034 | Pass |
| Stainless Steel | 0.001 (0.025) | 1.10 × 10⁻¹¹ | 0.161 | Pass |
| Nitinol | 0.002 (0.051) | 5.76 × 10⁻¹¹ | 0.540 | Pass |
| Stainless Steel | 0.002 (0.051) | 1.61 × 10⁻¹⁰ | 2.579 | Pass |
| Nitinol | 0.003 (0.076) | 2.50 × 10⁻¹⁰ | 2.732 | Pass |
| Stainless Steel | 0.003 (0.076) | 7.45 × 10⁻¹⁰ | 13.058 | Pass |
| Nitinol | 0.004 (0.102) | 7.79 × 10⁻¹⁰ | 8.633 | Pass |
| Stainless Steel | 0.004 (0.102) | 2.04 × 10⁻⁹ | 21.077 | Fail |
| Nitinol | 0.005 (0.127) | 2.45 × 10⁻⁹ | 41.271 | Fail |
| Stainless Steel | 0.005 (0.127) | 4.39 × 10⁻⁹ | 100.758 | Fail |

The results of the experiment indicate that a flexural rigidity of less than 2.04×10⁻⁹ kN-m², and a critical buckling load of less than 21.08 grams-force allows for atraumatic advancement of a catheter device in the sub-retinal space.

### Example 3

Enucleated human and rabbit cadaver eyes were used to evaluate catheter access to the sub-retinal space. The eyes were prepared by removing the muscles, conjunctiva, and tenons. Both ab-interno and ab-externo approaches as described herein were performed. A catheter device with distal shaft outer diameter of 200 microns and a bulbous distal tip with diameter of 275 microns was used. The distal shaft was comprised of a polyether block amide tube with a durometer of 63 Shore D (Pebax 6333, Arkema Inc). The catheter had a measured average flexural rigidity in bending of 1.49×10⁻¹⁰ kN*m² and average critical load in buckling of 8.0 grams force. The distal shaft terminated proximally in a polymer hub with two proximal elements. The catheter device incorporated an 85 micron (0.003 inch) plastic optical fiber in the lumen extending to the distal tip which was connected to a 0.25 mm (0.010 inch) fiberoptic in one proximal element of the catheter. The larger fiberoptic terminated in a fitting for connection to a 658 nm (red) laser diode illumination source (iLumin™, iScience Interventional Inc). The lumen of the distal shaft was in communication through the hub to the second proximal element, which was comprised of a polymer tube which terminated in a female Luer fitting for attachment of a standard syringe.

For the ab-interno approach, a tubular introducer comprised of thin walled polyimide tubing (Microlumen, Inc) with an inner diameter of 300 microns (0.012 inch), a wall thickness of 25 microns (0.001 inch) and a length of 29 mm (1.14 inch) was fabricated. The distal end of the introducer was angled to approximately 30° and the distal tip was cut in a bevel to aid in piercing the retinal tissues. The distal angle was formed by placing an appropriately shaped stainless steel wire into the polyimide tubing and then heating the tubing to set the shape.

The eyes were prepared by pars plana placement of a 25 gauge sclerostomy port (Bausch & Lomb) for infusion of saline to maintain the shape of the globe, and a 23 gauge sclerostomy port (Bausch & Lomb) for introduction of the tubular introducer and catheter devices. An 8 mm diameter corneal trephine was used to remove the cornea to allow for an “open sky” view of the interior of the vitreous cavity. The iris and lens were then carefully removed. The tubular introducer was inserted through the sclerostomy port and advanced across the vitreous cavity. The distal curved tip was inserted through the peripheral retina and into the sub-retinal space with the curvature directed toward the posterior.

The catheter device was placed into the introducer and advanced. Under direct visualization, the illuminated beacon tip of the catheter was seen to advance in the sub-retinal space to the posterior pole. An injection of 0.1% fluorescein was made through the catheter lumen to confirm position of the catheter in the sub-retinal space and the ability to administer substances to the sub-retinal space. The catheter was then withdrawn through the introducer and the introducer removed from the globe.

For the ab-externo approach, a viscodissection cannula was used to create a small opening through the choroid using high viscosity sodium hyaluronate viscoelastic. The viscodissection cannula was fabricated by adhesively bonding (Loctite 4305, Loctite Corp) a polyimide tube (Microlumen, Inc) with an inner diameter of 64 um (0.0025 inch) and outer diameter of 89 microns (0.0035 inch) into a blunt 31 gauge hypodermic needle (Cadence Sciences, Inc). The adhesive was used to create a bulbous distal tip of 360 microns diameter. The viscodissection cannula was attached via a short infusion line to a screw driven syringe (ViscoInjector™, iScience Interventional) containing Healon® GV (Abbot Medical Optics).

A scleral incision was made to access the suprachoroidal space and expose the choroid. The viscodissection cannula was primed with Healon. The distal tip of the cannula was placed in contact with the choroidal surface and the viscoelastic flow was started by advancing the syringe screw. Light pressure against the choroid was used while the viscoelastic flow dissected the tissues. High frequency (80 Mhz) ultrasound imaging (iUltrasound™, iScience Interventional Inc) was used to confirm that a small opening was dissected through the choroid but not through the retina, and a pocket of viscoelastic was left in the sub-retinal space.

A catheter, as described in Example 2 above, was inserted through the choroidal fistula and into the subretinal space. The illuminated beacon tip was observed trans-sclerally as the catheter tip was advanced to the posterior pole. The high frequency ultrasound system was used to verify the location of the catheter in the subretinal space.

### Example 4

Testing of ab-interno and ab-externo access to the sub-retinal space was performed in live animal studies using a rabbit model. The studies were performed under protocol approved by the Institutional Animal Care and Use Committee (IACUC). The rabbits were anesthetized per protocol, draped and prepared for ophthalmic surgery.

To test the ab-interno approach in a rabbit eye, two small access pars plana incisions were made with an MVR blade for infusion and vitrectomy access, and one 23 gauge sclerostomy port was placed in the pars plana for placement of the tubular introducer and catheter. After a vitrectomy was performed, a curved tip, thin-walled introducer was placed through the 23 gauge port. The introducer was fabricated as in Example 2. The introducer was advanced across the globe and the distal tip was inserted into the peripheral retinal to allow access by the catheter to the sub-retinal space. The catheter tip was placed through the introducer into the sub-retinal space and advanced toward the macular region.

In the ab-externo approach, a small incision was made through the conjunctiva and sclera to expose the choroid along the posterior pars plana. A small incision was made in the choroid and the microcatheter was inserted through the choroid and under the sensory retina without perforation of the retina. The catheter was advanced to the posterior pole and an injection of an aqueous solution was made. The catheter was withdrawn and incision sutured closed. Imaging via optical coherence tomography (OCT) showed a distinct tract in the subretinal space leading to a retinal bleb created by the injectate in the sub-retinal space.

### Example 5

Viscodissection cannulas with a protruding element according to the invention were fabricated. A 30 gauge by 0.5 inch (12.7 mm) dispensing cannula (EFD, Inc) comprised of a stainless steel main shaft with a polyethylene female Luer connector on the proximal end was obtained. The distal 0.1 inch (2.5 mm) was bent at a 45 degree angle from the axis. A 0.001 inch (25 micron) diameter type 304 stainless steel wire (Ft. Wayne Metals) was placed into the lumen of a polyimide tube (Accelent, Inc.) approximately 0.4 inch (10 mm) long with inner diameter of 0.003 inch (75 micron) and an outer diameter of 0.004 inch (100 micron). The wire was folded over 180 degrees, the bend was brought into contact with one edge of the polyimide tube and the wire adhesively bonded to the outside of the tube (the proximal end). The wire was trimmed next to the bond so that the untrimmed wire end extended from the opposite end of the polyimide tube. The proximal end of the polyimide tube was then inserted into the distal end of the 30 gauge cannula. The polyimide tube was adhesively bonded so that the tube extended from the distal end of the 30 gauge cannula for 500 microns (0.020 inch). The stainless wire was then trimmed so that it extended beyond the polyimide tubing for a distance of 50 microns (0.002 inch).

### Example 6

Viscodissection cannulas according to Example 4 were fabricated and then packaged into sterile barrier peel pouches and sterilized using minimum 25 kGy of gamma radiation. The devices were used in live animal surgery in both rabbit and porcine models to create an access route to the sub-retinal space. The conjunctiva was incised and retracted to allow access to the scleral surface. An incision approximately 2 mm (0.8 inch) long was made in the sclera at a point between 6.5 mm (0.26 inch) and 7.5 mm (0.3 inch) posterior to the limbus. The scleral incision opening was maintained using a wire micro-retractor to expose the choroidal surface.

A viscodissection cannula was attached to a syringe of viscoelastic (Healon®, Abbott Medical Optics, Inc.) and the plunger depressed to start the flow of viscoelastic from the cannula. Under magnification with the surgical microscope, the distal tip of the cannula was brought in contact with the choroid. While expressing viscoelastic, the protruding element was placed between choroidal blood vessels and slight pressure was used to pierce the choroid layer. Viscoelastic was continued to be expressed during the formation of the choroidal fistula, with the viscoelastic entering the sub-retinal space, separating the retina from the RPE and creating a sub-retinal bleb. The presence of the bleb as well as the completion of the procedure without penetrating the retina was confirmed using an indirect ophthalmoscope, which allows for direct viewing of the peripheral retina in an intact eye.

A catheter according to Example 2 was used to enter the choroidal opening at a low angle (parallel to the tissue planes) and then advanced to the posterior pole. The location of the catheter tip in the sub-retinal space and at the correct location was confirmed by indirect ophthalmoscope. Injections of 0.1% fluorescein were made. After euthanasia, the eyes were enucleated and dissected and the presence of fluorescein in the sub-retinal space was confirmed visually.

1. A device for access to the subretinal space of an eye comprising:
a catheter having a proximal end and distal end, said distal end comprising an atraumatic tip having a smooth surface;
said catheter having a distal end of 25 to 40 mm length, with flexural rigidity in bending and a response to critical buckling load sufficient to allow flexing of said catheter in the eye without causing substantial tissue trauma or distension of local tissue.

2. The device according to claim 1 wherein said catheter has a round profile of a maximum diameter of at least 200 microns.

3. The device according to claim 1 where said flexural rigidity in bending is less than 2.04×10⁻⁹ kN-m².

4. The device according to claim 1 wherein said response to critical buckling load is less than 21.08 grams-force.

5. The device according to claim 1 comprising an illuminated beacon tip.

6. The device according to claim 1 wherein the surface of said catheter is lubricious.

7. The device according to claim 1 wherein said catheter comprises external depth markings.

8. The device of claim 1 where said catheter comprises a region adjacent to said atraumatic tip having lower flexural rigidity than said catheter to allow said tip to flex upon encountering an obstruction during insertion of said catheter into the eye.

9. A tubular shaft introducer characterized by a primary shaft axis and comprising a distal end disposed at an angle to said primary shaft axis, said shaft comprising a lumen of sufficient diameter to accommodate a catheter having a smooth surface.

10. The introducer according to claim 9 wherein said angle is in the range of 20° to 9°.

11. The introducer according to claim 9 wherein said distal end is of a length of 2 to 10 mm.

12. The introducer according to claim 9 wherein said primary shaft axis is of a length of 25 to 40 mm.

13. A cannula device having a proximal end and distal end, said device comprising a bulbous distal tip and a lumen, said bulbous distal tip being of sufficient size to atraumatically dissect the choroid of an eye to access the subretinal space for injecting through said lumen a viscoelastic substance to create an opening through the choroid to access the subretinal space of the eye.

14. The cannula device according to claim 13 wherein said bulbous tip has a diameter of at least 200 microns.

15. The cannula device according to claim 13 having a protruding element at said distal tip.

16. The cannula device according to claim 15 wherein said protruding element protrudes from 10 to 100 microns from said distal tip.

17. The cannula device according to claim 15 wherein said protruding element comprises a fiber optic.

18. A method for catheterizing the sub-retinal space adjacent to the macula of an eye by introducing a catheter into the sub-retinal space in an area of peripheral retina by advancing a tip of said catheter in the sub-retinal space toward the macula.

19. The method according to claim 18 wherein said catheter has a proximal end and distal end and said distal end comprises said tip advanced in the sub-retinal space, said distal end comprising an atraumatic tip having a smooth surface;
said catheter having flexural rigidity in bending and a response to critical buckling load sufficient to allow flexing of said catheter in the eye without causing substantial tissue trauma or distension of local tissue.

20. The device according to claim 19 where said flexural rigidity in bending is less than 2.04×10⁻⁹ kN-m².

21. The device according to claim 19 wherein said response to critical buckling load is less than 21.08 grams-force.

22. The method according to claim 18 wherein the catheter is placed in the sub-retinal space from an ab-externo approach, by dissecting the sclera to access the suprachoroidal space then dissecting the choroid to create an opening to the sub-retinal space.

23. The method according to claim 22 wherein the dissection of choroid is performed using a cannula device with a proximal end, a bulbous distal tip and a lumen, said bulbous distal tip being of sufficient size to dissect the choroid of an eye to create an opening through the choroid by injecting through said lumen a viscoelastic substance for the purpose of introducing said catheter into the sub-retinal space of the eye.

24. The method according to claim 22 wherein said bulbous tip has a diameter of at least 200 microns.

25. The method according to claim 23 wherein said cannula device has a protruding element at said distal tip.

26. The method according to claim 23 wherein said protruding element protrudes from 10 to 100 microns from said distal tip.

27. The method according to claim 25 wherein said protruding element comprises a wire or fiber optic.

28. A method for catheterizing the sub-retinal space adjacent to the macula of an eye by introducing a tubular shaft into the sub-retinal space in an area of peripheral retina, said shaft characterized by a primary shaft axis and comprising a distal end disposed at an angle to said primary shaft axis, said shaft comprising a lumen of sufficient diameter to accommodate a catheter having a smooth surface.

29. The method according to claim 28 wherein said angle is in the range of 20° to 90°.

30. The method according to claim 28 wherein said distal end is of a length of 2 to 10 mm.

31. The method according to claim 28 wherein said primary shaft axis is of a length of 25 to 40 mm.

32. The method according to claim 18 wherein the catheter is placed in the sub-retinal space from an ab-interno approach, by forming an opening in the peripheral retina ab-interno, to access the sub-retinal space, placing the tip of the catheter through the retinotomy and advancing it posteriorly, administering therapeutic substances, withdrawing the catheter and sealing the opening in the peripheral retina.
